(19) United States
(12) Patent Application Publication
(10) Pub. No.: US 2018/0372598 A1
(43) Pub. Date: Dec. 27, 2018

(54) METHOD, EXTRACTION INSTRUMENT, DISPENSING INSTRUMENT AND KIT FOR PRETREATING AN IN PARTICULAR BIOLOGICAL SAMPLE

(71) Applicant: Boehringer Ingelheim Vetmedica GmbH, Ingelheim am Rhein (DE)

(72) Inventors: Christoph Weber, Hannover (DE); Heinz Ulrich Gerhard Schoeder, Isernhagen (DE)

(73) Assignee: Boehringer Ingelheim Vetmedica GmbH, Ingelheim am Rhein (DE)

(21) Appl. No.: 16/009,253

(22) Filed: Jun. 15, 2018

(30) Foreign Application Priority Data
Jun. 21, 2017 (DE) 10 2017 005 864.4

(51) Int. Cl. G01N 1/40; C12N 15/10

(52) U.S. Cl. CPC G01N 1/40; G01N 2001/4088; C12N 15/1003; G01N 1/4077

(57) ABSTRACT

A method, an extraction instrument and a dispensing instrument for pretreating, in particular, a biological sample for, in particular, molecular-biological testing of the sample, the extraction instrument being sealed or able to be sealed by means of compression, and/or the sample being introduced or able to be introduced into the dispensing instrument through a piston of the dispensing instrument and/or being filtered or able to be filtered in the dispensing instrument. Furthermore, a kit for pretreating, in particular, a biological sample for, in particular, molecular-biological testing of the sample which includes a receiving instrument for receiving the sample, an extraction instrument, a treatment agent for treating the sample and a dispensing instrument.

### BACKGROUND OF THE INVENTION

### Field of the Invention

The present invention relates to a method for pretreating, in particular, a biological sample, to an extraction instrument for extracting, in particular, a biological sample, to a dispensing instrument for dispensing, in particular, a biological sample, and to a kit for pretreating, in particular, a biological sample.

Preferably, the present invention deals with analyzing and/or testing a sample, in particular, from a human or animal, particularly preferably for analytics and diagnostics, for example, with regard to the presence of diseases and/or pathogens and/or for determining blood counts, antibodies, hormones, steroids or the like. Therefore, the present invention is in particular within the field of bioanalytics. A food sample, environmental sample or another sample may optionally also be tested, in particular for environmental analytics or food safety and/or for detecting other substances.

The present invention deals, in particular, with what are known as point-of-care systems, i.e., in particular with mobile systems, devices and other apparatuses, and deals with methods for carrying out pretreatments and/or tests on a sample at the sampling site and/or independently and/or away from a central laboratory or the like. Preferably, point-of-care systems can be operated autonomously of and/or independently from a mains network for supplying electrical power.

Preferably, at least one analyte or target analyte of a sample can be determined, identified or detected by means of point-of-care systems of this kind. In particular, the sample can be tested for qualitatively or quantitatively determining at least one analyte, for example in order for it to be possible to detect or identify a disease and/or pathogen.

Within the meaning of the present invention, analytes are in particular nucleic-acid sequences, in particular DNA sequences and/or RNA sequences, or proteins, in particular antigens and/or antibodies. In particular, by means of point-of-care systems, nucleic-acid sequences can be determined, identified or detected as analytes of a sample, or proteins can be determined, identified or detected as analytes of the sample.

The present invention preferably deals with systems, devices and other apparatus for preparing for and/or carrying out a nucleic-acid assay for detecting or identifying a nucleic-acid sequence or a protein assay for detecting or identifying a protein.

### Description of Related Art

U.S. Pat. No. 5,096,669 discloses a point-of-care system for testing a biological sample, in particular a blood sample. The system comprises a single-use cartridge and an analysis device. Once the sample has been received, the cartridge is inserted into the analysis device in order to carry out the test. The cartridge comprises a microfluidic system and a sensor apparatus comprising electrodes, which apparatus is calibrated by means of a calibration liquid and is then used to test the sample.

Furthermore, International Patent Application Publication WO 2006/125767 A1 and corresponding U.S. Pat. No. 9,110,044 B2 disclose a point-of-care system for integrated and automated DNA or protein analysis, comprising a single-use cartridge and an analysis device for fully automatically processing and evaluating molecular-diagnostic analyses using the single-use cartridge. The cartridge is designed to receive a sample, in particular blood, and in particular allows cell disruption, PCR and detection of PCR amplification products, which are bonded to capture molecules and provided with a label enzyme, in order for it to be possible to detect bonded PCR amplification products or nucleic-acid sequences as target analytes in what is known as a redox cycling process.

Once a sample has been taken, said sample usually has to be pretreated or prepared for the subsequent test. For example, in order to test analytes of a sample, it is necessary for the analytes to first be extracted from or dissolved out of the sample, in particular for the analytes to be activated by cell disruption and/or for the sample to be filtered.

In point-of-care systems, the pretreated or prepared sample is usually received in a cartridge, and the cartridge is then inserted, together with the sample, into an analysis device, in order to carry out the test.

### SUMMARY OF THE INVENTION

The object of the present invention is to provide an improved method for pretreating a sample, an improved extraction instrument for extracting a sample, an improved dispensing instrument for dispensing a sample to a cartridge, and an improved kit for pretreating a sample, simple, reliable, cost-effective, hygienic and/or rapid pretreatment or preparation of the sample for, in particular, molecular-biological testing preferably being made possible or facilitated, and/or it being possible, as a result, to directly test the sample, preferably without any further preparation of the sample, in particular by means of a point-of-care system.

The above object is achieved by a method, by an extraction instrument, by a dispensing instrument, or by a kit as described herein.

Within the meaning of the present invention, an “instrument” is preferably a tool, apparatus or device used for receiving, pretreating, preparing and/or dispensing a sample which is to be analyzed. Such instruments are particularly preferably manually operated or operable by a user. However, also other solutions are also possible here.

In the proposed method, a sample, particularly preferably saliva, mucosa or the like, from animals, in particular pigs, is preferably pretreated for a subsequent, in particular molecular-biological test, sample components and/or analytes of the sample, in particular nucleic-acid sequences, preferably being extracted from or dissolved out of the sample and/or being activated, preferably by means of a treatment or extraction agent, in particular a lysis buffer, and/or being filtered, in particular in order to subsequently identify or detect, in particular electrochemically, the sample components and/or analytes, particularly preferably the nucleic-acid sequences, preferably in or by means of a cartridge.

In the proposed method, the sample is preferably first received or absorbed by a receiving element, such as a swab, and the receiving element is then inserted or introduced at least in part into an extraction instrument that is flexible, elastic, resilient, bendable and/or compressible at least in part.

In the extraction instrument, by means of a treatment or extraction agent, in particular a solvent, such as a lysis buffer, and/or by compressing or deforming the extraction instrument, the sample, the sample components and/or the analytes to be tested is/are separated from or dissolved out of the receiving element, in particular manually or without using any other instruments or apparatus, and/or is/are lysed and/or activated.

One aspect of the present invention is that, in order to separate the sample from the receiving element, the extraction instrument, in particular the inlet of the extraction instrument or a valve at the inlet of the extraction instrument, is sealed or closed by or during compression of the extraction instrument, in particular such that the sample and/or the treatment agent is prevented from leaving or spurting out of the extraction instrument during extraction.

Particularly preferably, by compressing or deforming the extraction instrument, in order to separate the sample from the receiving element, a valve of the extraction instrument is actuated or closed, in particular such that the sample and/or the treatment agent is prevented from leaving or spurting out of the extraction instrument.

This allows or facilitates a particularly simple, hygienic and/or reliable pretreatment, in particular extractions, of the sample. In particular, the proposed method prevents the sample from entering the surroundings in an inadvertent or uncontrolled manner, from contaminating the surroundings and/or from putting a user in danger during pretreatment and/or extraction.

According to another aspect of the present invention, which can also be implemented independently, the treatment agent is introduced into the extraction instrument, in particular via an outlet of the extraction instrument, in particular by means of a transfer instrument, in order to dissolve or lyse the sample, and/or the extraction instrument is rinsed with the treatment agent, and/or the in particular pretreated sample is removed, preferably together with at least some of the treatment agent, from the extraction instrument, in particular again via the outlet. This provides for very simple handling.

The sample is preferably delivered or dispensed from the transfer instrument directly to a dispensing instrument for filtering and/or to a cartridge for testing.

According to another aspect of the present invention, which can also be implemented independently, the sample, which is in particular extracted or obtained as mentioned above, is introduced, in particular by means of a transfer instrument, such as a syringe, from the extraction instrument into a dispensing instrument for dispensing the sample to a cartridge, and is filtered in the dispensing instrument, in particular while the sample is being dispensed to the cartridge. This allows or facilitates a particularly simple, rapid, hygienic and/or cost-effective pretreatment, in particular filtration, of the sample.

In the present invention, the term “extraction instrument” is preferably understood to mean a structural apparatus or instrument designed to extract or dissolve components and/or analytes of an in particular biological sample, preferably from a receiving element, such as a swab, and/or to activate said components and/or analytes, in particular by means of a treatment or extraction agent, such as a lysis buffer, and/or by having a mechanical effect on the receiving element.

Preferably, within the meaning of the present invention, an extraction instrument is designed to receive a receiving element, such as a swab, and/or, within the meaning of the present invention, an extraction instrument comprises an in particular elongate extraction chamber for receiving a receiving element, such as a swab.

Within the meaning of the present invention, an extraction instrument is preferably flexible and/or compressible at least in part and/or manually actuatable by a user, in particular in order to compress or wring out a receiving element, such as a swab. Within the meaning of the present invention, an extraction instrument preferably comprises an inlet and/or outlet the inlet in particular being closable during extraction, it preferably being possible for the extracted sample components and/or analytes to be removed via the outlet from the extraction chamber. More particularly preferably, within the meaning of the present invention, an extraction instrument is a container, a bag, a pouch, a funnel or the like.

The proposed extraction instrument preferably comprises an in particular elongate extraction chamber and a wall for defining or delimiting the extraction chamber in particular laterally and/or radially, the extraction instrument and/or the wall of the extraction instrument in particular being deformable, in particular compressible, at least in part, preferably such that the receiving element can be squeezed or wrung out in the extraction instrument or extraction chamber, and/or such that the sample components and/or the analytes of the sample can be separated from, in particular dissolved out of or eluted from, the receiving element.

One aspect of the present invention is that the extraction instrument comprises at least one valve, it being possible for the valve to be actuated, in particular closed, by compressing the extraction instrument and/or the wall, and/or a valve opening of the valve being arranged in the extraction chamber, in particular such that the valve opening can be closed by compressing the extraction instrument and/or the wall.

Particularly preferably, the valve and/or the preferably elongate valve body of the valve extends from the inlet of the extraction instrument into the extraction chamber, in particular such that the valve opening of the valve is arranged at least substantially centrally in the extraction chamber.

This allows or facilitates a particularly simple, cost-effective and/or stable construction of the extraction instrument and/or simple, rapid, hygienic and/or reliable handling of the extraction instrument and/or pretreatment of the sample.

Particularly preferably, the valve is designed as a film valve, and/or the valve comprises an at least substantially planar or flat valve body of which the cross section is preferably at least substantially elliptical, it preferably being possible for the valve body, in particular the axial end of the valve body, to be compressed by compressing the extraction instrument or the wall, and/or the valve body extending from the inlet of the extraction instrument into the extraction chamber, in particular such that the valve opening is arranged at least substantially centrally in the extraction chamber. This results in corresponding advantages.

According to another aspect of the present invention, which can also be implemented independently, the wall of the extraction instrument has a varying or different wall thickness. In particular, the wall of the extraction instrument comprises a first side part and a second side part, the first side part being more rigid, in particular thicker, than the second side part, in particular such that, when the extraction instrument is compressed, it is possible for the second side part to be more easily moved or pressed against or towards the first side part and/or against the valve opening or the valve body. This allows or facilitates simple handling or actuation of the extraction instrument. In particular, the amount of force required to actuate the extraction instrument is reduced.

Within the meaning of the present invention, the term “dispensing instrument” is preferably understood to mean a structural apparatus or instrument which is designed to introduce the in particular pretreated sample, which has been particularly preferably extracted and/or filtered, in a cartridge for the subsequent test of the sample.

Within the meaning of the present invention, a dispensing instrument is preferably (additionally) designed to pretreat or prepare a sample for the subsequent test of the sample.

In particular, within the meaning of the present invention, a dispensing instrument comprises a preferably at least substantially cylindrical housing and a piston, the piston being axially movable in the housing, in particular in order to dispense the sample. More particularly preferably, within the meaning of the present invention, a dispensing instrument is designed as a syringe or the like.

Within the meaning of the present invention, a “piston” is preferably a movable part of the dispensing instrument which fits tightly, in particular sealingly, into the housing of the dispensing instrument. The seal between the housing and the piston is thus preferably movable with the piston. However, it is also possible that the seal is stationary and the piston can slide through the seal. For example, the piston can be sealed at the top of the housing, in particular opposite of an outlet of the dispensing instrument.

The proposed dispensing instrument is preferably designed to dispense the sample, which has preferably been pretreated by means of the extraction instrument and/or the sample components and/or analytes that have preferably been extracted and/or activated by means of the extraction instrument, to a cartridge.

One aspect of the present invention is that the piston of the proposed dispensing instrument comprises a piston channel, the piston channel preferably extending axially through the piston and/or comprising or forming an inlet into the chamber of the dispensing instrument.

Particularly preferably, the in particular extracted sample can be introduced into the dispensing instrument or the chamber of the dispensing instrument via the piston or piston channel, and/or the piston comprises a connection for a transfer instrument, such as a syringe, in particular in order to introduce the sample into the dispensing instrument or into the chamber thereof.

This allows or facilitates particularly simple, reliable, hygienic and/or rapid handling and/or pretreatment of the sample. In particular, a construction of this kind allows fluid to flow axially through the dispensing instrument or for fluid to flow through the dispensing instrument in only one main flow direction. This is expedient for the pretreatment of the sample by means of the dispensing instrument, as explained in greater detail in the following.

According to another aspect of the present invention, which can also be implemented independently, the dispensing instrument comprises, in particular, a multilayer filter for filtering the sample, the filter being in particular designed for letting through the sample components and/or analytes, in particular nucleic-acid sequences, to be tested and/or for separating or filtering out undesired sample components, such as particles, impurities, proteins or the like.

Preferably, the filter is integrated in the dispensing instrument and/or arranged in the chamber or the housing of the dispensing instrument. Particularly preferably, the filter is arranged on or directly upstream of the outlet of the dispensing instrument, in particular such that the sample is pretreated or filtered in the dispensing instrument and/or during or immediately before dispensing. This results in corresponding advantages.

By using the proposed dispensing instrument, it is preferably possible to directly test the pretreated sample and/or to feed said sample to the cartridge, such that further preparation or filtration of the sample can be omitted.

The proposed kit for pretreating an in particular biological sample for an in particular molecular-biological test of the sample, in particular for detecting or identifying a nucleic-acid sequence, preferably comprises a receiving instrument comprising a receiving element, such as a swab, for receiving the sample, a proposed extraction instrument for extracting the sample from the receiving element, a treatment or extraction agent, in particular a lysis buffer, for treating the sample, and a proposed dispensing instrument for dispensing the sample to a cartridge. This results in corresponding advantages.

Within the meaning of the present invention, a kit is in particular a group, a system and/or a combination comprising a receiving instrument for receiving the sample, a proposed extraction instrument, a treatment or extraction agent, and/or a proposed dispensing instrument for dispensing the sample. The receiving instrument, the extraction instrument, the treatment agent and/or the dispensing instrument preferably each form a component of the kit. The components of the kit are preferably marketed as a group, in particular in a common container or the like. It is however also possible for the aforementioned components to form a group of separate components for joint use. A common or unifying component is preferably provided in the proposed kit, for example operating instructions, recommendations for use, references on the labelling of one or more of the components of the kit and/or a container, such as packaging or the like.

The above-mentioned aspects and features of the present invention and the aspects and features of the present invention that will become apparent from the claims and the following description can in principle be implemented independently from one another, but also in any combination or order.

Other aspects, advantages, features and properties of the present invention will become apparent from the following description of a preferred embodiment with reference to the accompanying drawings.

### BRIEF DESCRIPTION OF THE DRAWINGS

FIG. 1 is a schematic view of an analysis device and a cartridge received in the analysis device;

FIG. 2 is a schematic view of the cartridge;

FIG. 3 is a schematic section through the cartridge when said cartridge is being filled by means of a proposed dispensing instrument;

FIG. 4 is a schematic view of a proposed kit comprising a proposed extraction instrument, a proposed dispensing instrument, a receiving instrument and a transfer instrument;

FIG. 5 is a schematic section through the proposed extraction instrument;

FIG. 6 is a schematic section through the extraction instrument according to FIG. 5, rotated by 90°;

FIG. 7 is a schematic section through the dispensing instrument;

FIG. 8 is a schematic view of the extraction instrument together with an inserted receiving instrument when said extraction instrument is being connected to the transfer instrument;

FIG. 9 is a schematic view of the extraction instrument according to FIG. 8 together with a connected transfer instrument when the extraction instrument is being filled with a treatment agent;

FIG. 10 is a schematic view of the extraction instrument according to FIG. 9 when the sample is being pretreated and/or when the extraction instrument is being compressed;

FIG. 11 is a schematic view of the extraction instrument according to FIG. 10 when the pretreated sample is being removed from the extraction instrument by means of the transfer instrument;

FIG. 12 is a schematic view of the cartridge together with a connected dispensing instrument when the transfer instrument is being connected to the dispensing instrument; and

FIG. 13 is a schematic view of the cartridge according to FIG. 12 when the sample is being received by means of the dispensing instrument.

### DETAILED DESCRIPTION OF THE INVENTION

In the figures, which are only schematic and sometimes not to scale, the same reference signs are used for the same or similar parts and components, corresponding or comparable properties and advantages being achieved even if these are not repeatedly described.

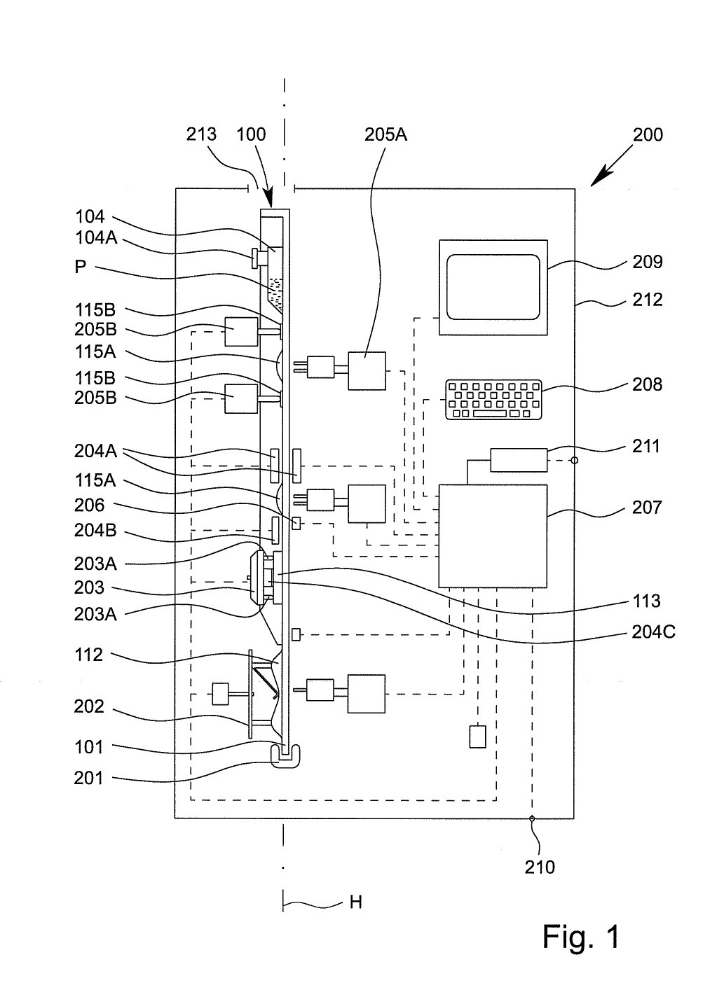

FIG. 1 is a highly schematic view of an apparatus or cartridge 100 in an analysis device 200 for an in particular molecular-biological test of an in particular biological sample P.

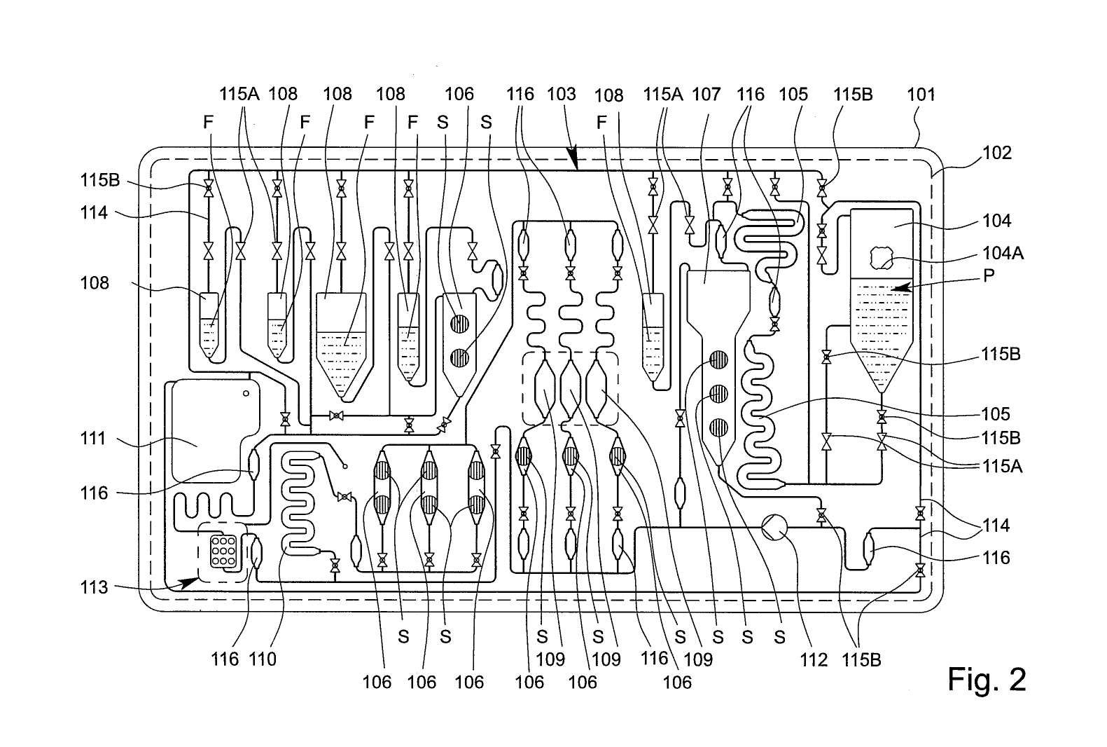

FIG. 2 is a schematic view of a preferred embodiment of the apparatus or cartridge 100 for testing the sample P. The apparatus or cartridge 100 in particular forms a handheld unit, and in the following is merely referred to as a cartridge 100.

The term “cartridge” is preferably understood to mean a structural apparatus or unit designed to receive, to store, to physically, chemically and/or biologically treat and/or prepare and/or to measure a sample, preferably in order to make it possible to detect, identify or determine at least one analyte, in particular a protein and/or a nucleic-acid sequence, of the sample.

A cartridge within the meaning of the present invention preferably comprises a fluid system having a plurality of channels, cavities and/or valves for controlling the flow through the channels and/or cavities.

In particular, within the meaning of the present invention, a cartridge is designed to be at least substantially planar and/or card-like, in particular is designed as a (micro)fluidic card and/or is designed as a main body or container that can preferably be closed and/or said cartridge can be inserted and/or plugged into an analysis device when it contains the sample.

The term “analysis device” is preferably understood to mean a structural apparatus designed to chemically, biologically and/or physically test and/or analyse a sample or analysis sample or a component thereof, in particular in order for it to be possible to directly and/or indirectly detect or identify a disease and/or pathogen. An analysis device within the meaning of the present invention is in particular a portable or mobile device designed in particular to immediately or directly test and/or analyze the sample, in particular on site and/or in the vicinity of the sampling site and/or away from a central laboratory.

The term “sample” is preferably understood to mean the sample material to be tested, which is in particular taken from a human or animal. In particular, within the meaning of the present invention, a sample is a fluid, such as mucosa, saliva, blood, urine or another liquid, preferably from a human or animal, or a component thereof. Within the meaning of the present invention, a sample may be pretreated or prepared, or may come directly from a human or animal or the like, for example.

A sample within the meaning of the present invention preferably contains one or more sample components or analytes to be tested, it preferably being possible for the analytes to be identified and/or detected, in particular qualitatively and/or quantitatively determined. Particularly preferably, within the meaning of the present invention, a sample has target nucleic-acid sequences as the analytes, in particular target DNA sequences and/or target RNA sequences, and/or target proteins as the analytes, in particular target antigens and/or target antibodies. Particularly preferably, at least one disease and/or pathogen can be detected or identified in the sample by qualitatively and/or quantitatively determining the analytes.

Preferably, the analysis device 200 controls the testing of the sample P in particular in or on the cartridge 100 and/or the analysis device 200 is used to evaluate the testing and/or to collect, to process and/or to store measured values from the test.

By means of the analysis device 200 and/or by means of the cartridge 100 and/or using the method for testing the sample P, an analyte of the sample P, or particularly preferably a plurality of analytes of the sample P, can preferably be determined, identified or detected. Said analytes are in particular detected, identified and/or measured not only qualitatively, but particularly preferably also quantitatively.

Therefore, the sample P can be tested for qualitatively or quantitatively determining at least one analyte, for example in order for it to be possible to detect or identify a disease and/or pathogen or to determine other values, which are important for diagnostics, for example.

The cartridge 100 is preferably at least substantially planar, flat, plate-shaped and/or card-like.

The cartridge 100 preferably comprises an in particular at least substantially planar, flat, plate-shaped and/or card-like main body or support 101, the main body or support 101 in particular being made of and/or injection-molded from plastics material, particularly preferably polypropylene.

The cartridge 100 preferably comprises at least one film or cover 102 for covering the main body 101 and/or cavities and/or channels formed therein at least in part, in particular on the front, and/or for forming valves or the like, as shown by dashed lines in FIG. 2.

The cartridge 100 and/or the main body 101 thereof, in particular together with the cover 102, preferably forms and/or comprises a fluidic system 103, referred to in the following as the fluid system 103.

The cartridge 100, the main body 101 and/or the fluid system 103 are preferably at least substantially vertically oriented in the operating position and/or during the test, in particular in the analysis device 200, as shown schematically in FIG. 1. In particular, the main plane H or surface extension of the cartridge 100 thus extends at least substantially vertically in the operating position.

The cartridge 100 and/or the fluid system 103 preferably comprises a plurality of cavities, in particular at least one receiving cavity 104, at least one metering cavity 105, at least one intermediate cavity 106, at least one mixing cavity 107, at least one storage cavity 108, at least one reaction cavity 109, at least one intermediate temperature-control cavity 110 and/or at least one collection cavity 111, the cavities preferably being fluidically interconnected by a plurality of channels.

Within the meaning of the present invention, channels are preferably elongate forms for conducting a fluid in a main flow direction, the forms preferably being closed transversely, in particular perpendicularly, to the main flow direction and/or longitudinal extension, preferably on all sides.

In particular, the main body 101 comprises elongate notches, recesses, depressions or the like, which are closed at the side by the cover 102 and form channels within the meaning of the present invention.

Within the meaning of the present invention, cavities or chambers are preferably formed by recesses, depressions or the like in the cartridge 100 or main body 101, which are closed or covered by the cover 102, in particular at the side. The volume or space enclosed by each cavity is preferably fluidically linked, in particular to the fluid system 103, by means of channels.

In particular, within the meaning of the present invention, a cavity comprises at least two openings for the inflow and/or outflow of fluids.

Within the meaning of the present invention, cavities preferably have a larger diameter and/or flow cross section than channels, preferably by at least a factor of 2, 3 or 4. In principle, however, cavities may in some cases also be elongate, in a similar manner to channels.

The cartridge 100 and/or the fluid system 103 also preferably comprises at least one pump apparatus 112 and/or at least one sensor arrangement or sensor apparatus 113.

The reaction cavity/cavities 109 is/are preferably designed to allow a substance located in the reaction cavity 109 to react when an assay is being carried out, for example, by being linked or coupled to apparatus or modules of the analysis device 200.

The reaction cavity/cavities 109 is/are used in particular to carry out an amplification reaction, in particular PCR, or several, preferably different, amplification reactions, in particular PCRs. It is preferable to carry out several, preferably different, PCRs, i.e., PCRs having different primer combinations or primer pairs, in parallel and/or independently and/or in different reaction cavities 109.

The amplification products, target nucleic-acid sequences and/or other portions of the sample P produced in the one or more reaction cavities 109 can be conducted or fed to the connected sensor arrangement or sensor apparatus 113, in particular by means of the pump apparatus 112.

The sensor arrangement or sensor apparatus 113 is used in particular for detecting or identifying, particularly preferably qualitatively and/or quantitatively determining, the analyte or analytes of the sample P, in this case particularly preferably the target nucleic-acid sequences and/or target proteins as the analytes. Alternatively, or additionally, however, other values may also be collected or determined.

The cartridge 100, the main body 101 and/or the fluid system 103 preferably comprise a plurality of channels 114 and/or valves 115, as shown in FIG. 2.

By means of the channels 114 and/or valves 115, the cavities 104 to 111, the pump apparatus 112 and/or the sensor arrangement or sensor apparatus 113 can be temporarily and/or permanently fluidically interconnected and/or fluidically separated from one another, as required and/or optionally or selectively, in particular, such that they are controlled by the analysis device 200.

The cavities 104 to 111 are preferably each fluidically linked or interconnected by a plurality of channels 114. Particularly preferably, each cavity is linked or connected by at least two associated channels 114, in order to make it possible for fluid to fill, flow through and/or drain from the respective cavities as required.

The receiving cavity 104 preferably comprises a connection 104A for introducing the sample P. In particular, the sample P may for example be introduced into the receiving cavity 104 and/or cartridge 100 via the connection 104A by means of a pipette, syringe or other instrument, as explained in greater detail in the following.

The cartridge 100 is preferably designed as a microfluidic card and/or the fluid system 103 is preferably designed as a microfluidic system. In the present invention, the term “microfluidic” is preferably understood to mean that the respective volumes of individual cavities, some of the cavities or all of the cavities 104 to 111 and/or channels 114 are, separately or cumulatively, less than 5 ml or 2 ml, particularly preferably less than 1 ml or 800 μl, in particular less than 600 μl or 300 μl, more particularly preferably less than 200 μl or 100 μl.

Particularly preferably, a sample P having a maximum volume of 5 ml, 2 ml or 1 ml can be introduced into the cartridge 100 and/or the fluid system 103, in particular the receiving cavity 104.

Reagents and liquids which are preferably introduced or provided before the test in liquid form as liquids or liquid reagents F and/or in dry form as dry reagents S are required for testing the sample P, as shown in the schematic view according to FIG. 2.

Furthermore, other liquids F, in particular in the form of a wash buffer, solvent for dry reagents S and/or a substrate, for example in order to form detection molecules and/or a redox system, are also preferably required for the test, the detection process and/or for other purposes, and are in particular provided in the cartridge 100, i.e., are likewise introduced before use, in particular before delivery.

The cartridge 100 preferably contains all the reagents and liquids required for pretreating the sample P and/or for carrying out the test or assay, in particular for carrying out one or more amplification reactions or PCRs, and therefore, particularly preferably, it is only necessary to receive the optionally pretreated sample P.

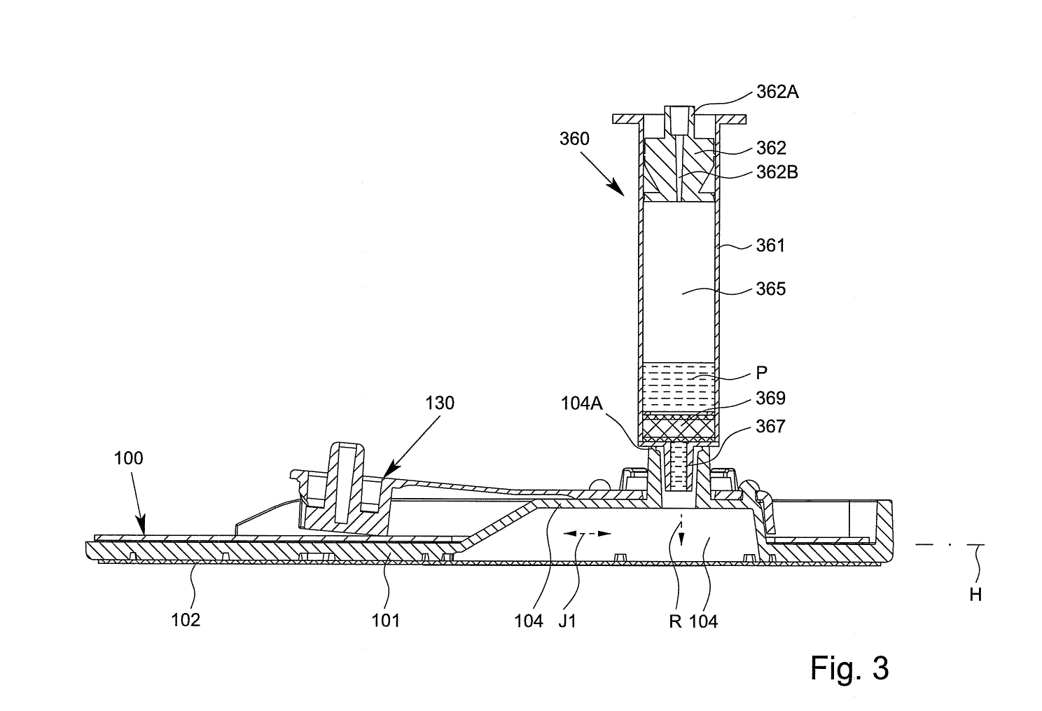

The connection 104A of the receiving cavity 104 can be closed after the sample P has been received. The cartridge 100 preferably comprises a closure element 130 for this purpose, as shown in FIG. 3.

In particular, the connection 104A can be closed in a liquid-tight and particularly preferably also gas-tight manner by the closure element 130. In particular, a closed fluid circuit can thus be formed, with the receiving cavity 104 being included. In particular, once the assigned valves 115A at an inlet, outlet and/or intermediate connection of the receiving cavity 104 have been opened, the receiving cavity 104 thus forms part of the fluid system 103 of the cartridge 100, wherein the fluid system is preferably closed or can be closed by the closure element 130.

The closure element 130 or the closure part thereof closes the receiving cavity 104 or the connection 104A thereof preferably in a permanent manner, i.e., it preferably cannot be released again. The connection 104A therefore preferably cannot be reopened after it has been closed.

FIG. 3 shows the cartridge 100 together with a connected dispensing instrument 360, but before the receiving cavity 104 is actually filled with the sample P or before said sample P is actually fed to said cavity 104.

The dispensing instrument 360 is preferably designed to dispense the pretreated sample P to the cartridge 100, as explained in greater detail in the following.

In the state shown, the dispensing instrument 360 is preferably connected to and/or plugged into the connection 104A by means of a connecting tip or connecting piece, particularly preferably in such a way that a vent or slots formed thereby of the connection 104A remain open so that, when the receiving cavity 104 is filled (in part) with the sample P, gas or air can escape from the receiving cavity 104 to the outside through the vent. In this regard, it is noted that, in the delivery state, the valves 115A assigned to the receiving cavity 104 are all closed, and the fluid system 103 is thus closed off from the receiving cavity 104 such that displaced air can escape only through the connection 104A and/or the vent that is particularly preferably provided. However, other structural solutions are in principle also possible.

The main direction R when filling the cartridge 100 with the sample P is shown schematically in FIG. 3. This main direction R extends in the opposite direction from the main opening direction of the connection 104A.

The main direction R preferably extends transversely and/or perpendicularly to a longitudinal extension J1 of the receiving cavity 104 and/or the main plane H of the cartridge 100.

Specifically, the receiving cavity 104 is designed such that the longitudinal extension L thereof extends at least substantially in the vertical direction in the operating position of the cartridge 100.

Specifically, as already explained, the plate plane or main plane H of the cartridge 100, as shown in FIGS. 1 and 3, is oriented at least substantially vertically during use.

After the receiving cavity 104 has been filled with the sample P, the dispensing instrument 360 is removed and the connection 104A is closed by the closure element 130 and/or the closure part thereof being placed onto the connection 104A in order to sealingly or tightly close said connection.

In the closed state, the closure element 130 or the closure part thereof is preferably sealingly or tightly held against or on the connection 104A in a latching, interlocking or form-fitting manner, in the example shown in particular by means of one or more retaining arms or elements which are in particular arm-like and/or which comprise or form one or more latching projections.

Once the sample P has been introduced into the receiving cavity 104 and the connection 104A has been closed, the cartridge 100 can be inserted into and/or received in the proposed analysis device 200 in order to test the sample P, as shown in FIG. 1.

The analysis device 200 preferably comprises a mount or receptacle 201 for mounting and/or receiving the cartridge 100.

Preferably, the cartridge 100 is fluidically, in particular hydraulically, separated or isolated from the analysis device 200. In particular, the cartridge 100 forms a preferably independent and in particular closed or sealed fluidic or hydraulic system 103 for the sample P and the reagents and other liquids. In this way, the analysis device 200 does not come into direct contact with the sample P and can in particular be reused for another test without being disinfected and/or cleaned first.

It is however provided that the analysis device 200 is connected or coupled mechanically, electrically, thermally and/or pneumatically to the cartridge 100.

In particular, the analysis device 200 is designed to have a mechanical effect, in particular for actuating the pump apparatus 112 and/or the valves 115, and/or to have a thermal effect, in particular for temperature-controlling the reaction cavity/cavities 109 and/or the intermediate temperature-control cavity 110.

In addition, the analysis device 200 can preferably be pneumatically connected to the cartridge 100, in particular in order to actuate individual apparatus, and/or can be electrically connected to the cartridge 100, in particular in order to collect and/or transmit measured values, for example, from the sensor apparatus 113 and/or from sensor portions 116.

The analysis device 200 preferably comprises a pump drive 202, the pump drive 202 in particular being designed for mechanically actuating the pump apparatus 112.

The analysis device 200 preferably comprises a connection apparatus 203 for in particular electrically and/or thermally connecting the cartridge 100 and/or the sensor arrangement or sensor apparatus 113.

As shown in FIG. 1, the connection apparatus 203 preferably comprises a plurality of electrical contact elements 203A, the cartridge 100, in particular the sensor arrangement or sensor apparatus 113, preferably being electrically connected or connectable to the analysis device 200 by the contact elements 203A.

The analysis device 200 preferably comprises one or more temperature-control apparatus 204 for temperature-controlling the cartridge 100 and/or having a thermal effect on the cartridge 100, in particular for heating and/or cooling, the temperature-control apparatus 204 (each) preferably comprising or being formed by a heating resistor or a Peltier element.

Preferably, individual temperature-control apparatuses 204, some of these apparatuses or all of these apparatuses can be positioned against the cartridge 100, the main body 101, the cover 102, the sensor arrangement, sensor apparatus 113 and/or individual cavities and/or can be thermally coupled thereto and/or can be integrated therein and/or can be operated or controlled in particular electrically by the analysis device 200. In the example shown, in particular the temperature-control apparatuses 204A, 204B and/or 204C are provided.

The analysis device 200 preferably comprises one or more actuators 205 for actuating the valves 115. Particularly preferably, different (types or groups of) actuators 205A and 205B are provided which are assigned to the different (types or groups of) valves 115A and 115B for actuating each of said valves, respectively.

The analysis device 200 preferably comprises one or more sensors 206. In particular, sensors 206 are assigned to the sensor portions 116 and/or are designed or provided for detecting liquid fronts and/or flows of fluid in the fluid system 103, the ambient temperature, internal temperature, atmospheric humidity, position, and/or alignment, for example by means of a GPS sensor, and/or the orientation and/or inclination of the analysis device 200 and/or the cartridge 100.

The analysis device 200 preferably comprises a control apparatus 207, in particular comprising an internal clock or time base for controlling the sequence of a test or assay and/or for collecting, evaluating and/or outputting or providing measured values in particular from the sensor apparatus 113, and/or from test results and/or other data or values.

The control apparatus 207 preferably controls or feedback controls the pump drive 202, the temperature-control apparatuses 204 and/or actuators 205, in particular taking into account or depending on the desired test and/or measured values from the sensor arrangement or sensor apparatus 113 and/or sensors 206.

Optionally, the analysis device 200 comprises an input apparatus 208, such as a keyboard, a touch screen or the like, and/or a display apparatus 209, such as a screen.

The analysis device 200 preferably comprises at least one interface 210, for example for controlling, for communicating and/or for outputting measured data or test results and/or for linking to other devices, such as a printer, an external power supply or the like. This may in particular be a wired or wireless interface 210.

The analysis device 200 preferably comprises a power supply 211 for providing electrical power, preferably a battery or an accumulator, which is in particular integrated and/or externally connected or connectable.

The analysis device 200 preferably comprises a housing 212, all the components and/or some or all of the apparatuses preferably being integrated in the housing 212. Particularly preferably, the cartridge 100 can be inserted or slid into the housing 212, and/or can be received by the analysis device 200, through an opening 213 which can in particular be closed, such as a slot or the like.

The analysis device 200 is preferably portable or mobile. Particularly preferably, the analysis device 200 weighs less than 25 kg or 20 kg, particularly preferably less than 15 kg or 10 kg, in particular less than 9 kg or 6 kg.

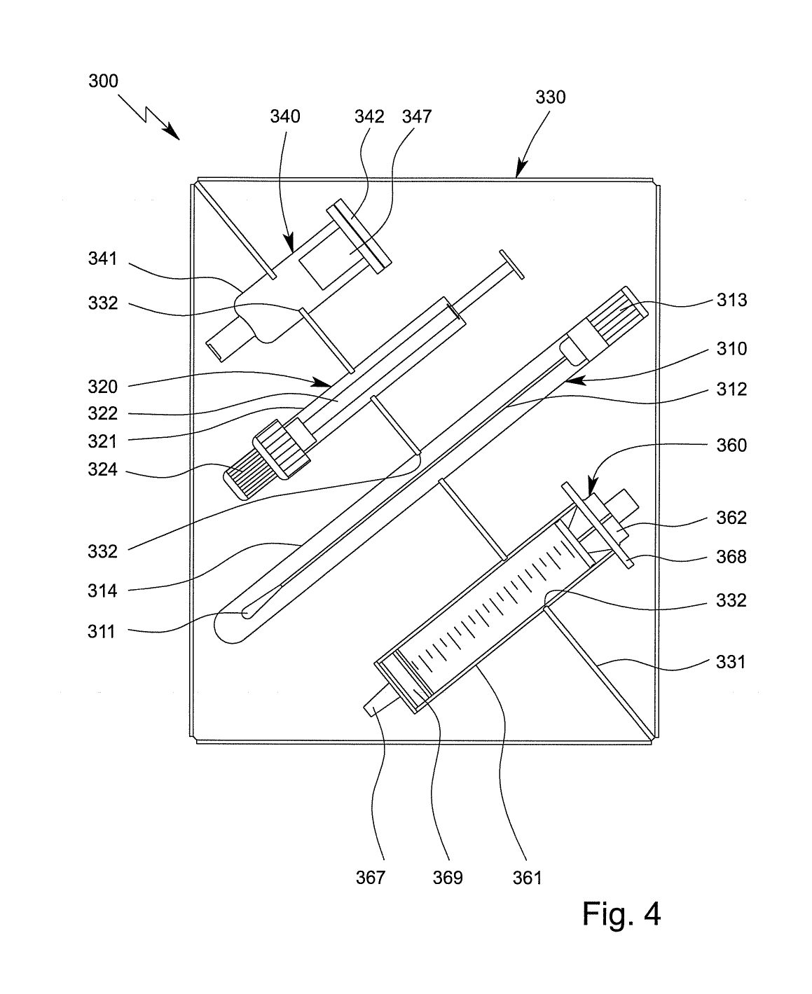

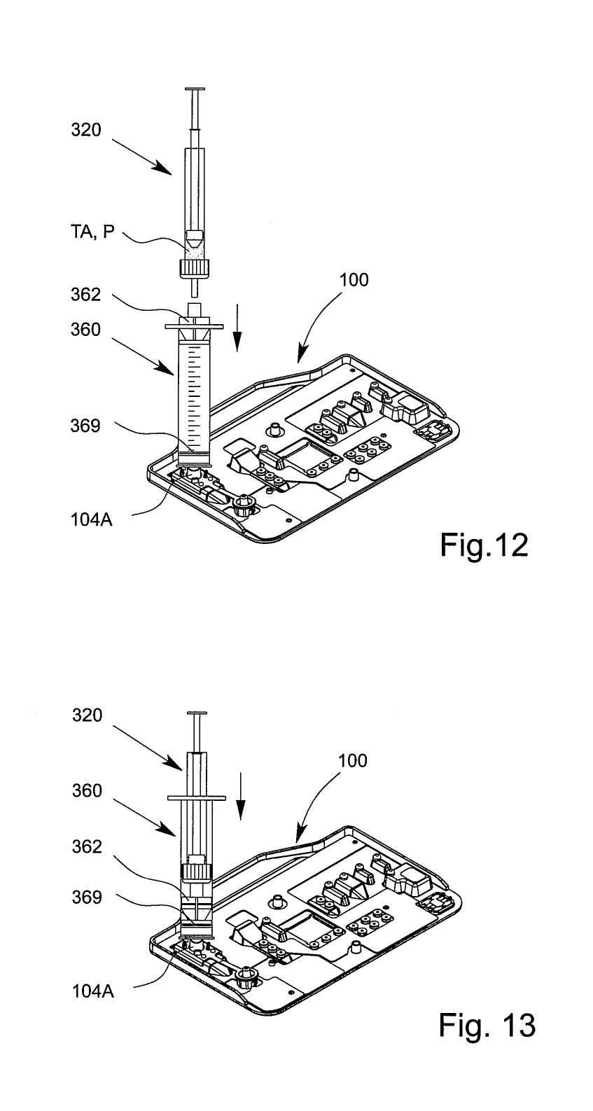

In the following, the pretreatment and/or preparation of the sample P for the test by means of the cartridge 100 and the analysis device 200 will be described, with reference to FIG. 4 to FIG. 13.

FIG. 4 shows a proposed kit 300 for pretreating the sample P for the test by means of the above-described cartridge 100 and/or the analysis device 200.

The kit 300 preferably comprises a receiving instrument 310, a transfer instrument 320, a container 330, an extraction instrument 340 and/or a dispensing instrument 360, the receiving instrument 310, the transfer instrument 320, the extraction instrument 340 and/or the dispensing instrument 360 preferably being arranged, marketed and/or transported as a group and/or in the common container 330.

The kit 300 optionally comprises a treatment or extraction agent TA, the treatment agent TA preferably being already introduced in the transfer instrument 320 and/or extraction instrument 340 in the delivery state of the kit 300. Alternatively, a further component or a separate container can be provided that contains the treatment agent TA.

The container 330 is preferably designed as packaging, a box, a case or the like.

The container 330 preferably comprises a mount 331 for the components of the kit 300, the receiving instrument 310, the transfer instrument 320, the extraction instrument 340 and/or the dispensing instrument 360 in particular being held in the container 330 by means of the mount 331 in a secure manner or in a manner in which they are mutually spaced and/or at least substantially unable to move relative to one another, in particular such that the components are prevented from slipping out of place and/or getting damaged, when being transported for example.

The container 330, in particular the mount 331, preferably comprises a plurality of receiving portions 332, a separate receiving portion 332 preferably being provided for each component of the kit 300.

Particularly preferably, the mount 331 and/or the receiving portions 332 is/are adapted to the geometry and/or size of the components of the kit 300, in particular such that the components are each held at least substantially without any play, clearance or backlash and/or in a form-fitting, interlocking and/or force-fitting manner.

In the embodiment shown, the mount 331 is formed by a diagonally extending raised portion or rib in the container 330, the receiving portions 332 preferably being formed by concave or at least substantially circular cut-outs in the mount 331. However, other structural solutions are also possible here, for example, those in which the mount 331 and/or the receiving portions 332 are formed by loops, hooks, brackets and/or depressions in a flexible material, such as foam.

The mount 331 and/or the receiving portions 332 allow a particularly space-saving arrangement of the components in the container 330.

Preferably, the components of the kit 300 are arranged, in the container 330, obliquely to the long sides of the container 330 and/or such that the longitudinal axes of said components are arranged at least substantially orthogonally to an (imaginary) diagonal of the container 330.

Particularly preferably, the longer components, in this case the receiving instrument 310 and the transfer instrument 320, are arranged centrally in the container 330, and/or the shorter components, in this case the extraction instrument 340 and the dispensing instrument 360, are arranged outwardly, off-set or eccentrically or in the edge region of the container 330.

The receiving instrument 310 is preferably designed to remove or receive a sample P from a human or animal, in particular a pig, and/or to provide a received sample P for the pretreatment of the sample P.

Preferably, the receiving instrument 310 comprises a receiving element 311, such as a swab, in particular a cotton swab, a connection element 312, in particular in the form of a thin rod, and a holding element or a handle 313, the receiving element 311 and the holding element 313 preferably being arranged at opposite axial ends of the receiving instrument 310 and/or being interconnected by means of the connection element 312.

Preferably, the receiving instrument 310 is elongate and/or the receiving instrument 310 is more than 3 cm or 10 cm long and/or less than 100 cm, 80 cm or 30 cm long.

Preferably, by means of the receiving instrument 310, a user of the kit 300 or receiving instrument 310 is able to remove sample material from a human or animal, in particular a pig, in particular without thereby coming into (direct) contact with the sample material or the human or animal.

Preferably, the receiving instrument 310 comprises an optional vessel 314, the receiving element 311 and/or the connection element 312 preferably being or being able to be arranged inside the vessel 314, and/or the holding element 313 forming or comprising a closure element or a cap of the vessel 314.

The transfer instrument 320 is preferably designed to transfer a fluid, in particular the treatment agent TA and/or the sample P, particularly preferably from the extraction instrument 340 into the dispensing instrument 360.

Particularly preferably, the transfer instrument 320 is designed to introduce the treatment agent TA into the extraction instrument 340, to remove the (pretreated) sample P from the extraction instrument 340, and/or to introduce the (pretreated) sample P into the dispensing instrument 360 and/or in particular into the cartridge 100 via the dispensing instrument 360.

Preferably, the transfer instrument 320 is designed as a syringe and/or the transfer instrument 320 comprises a preferably cylindrical housing 321, a piston 322, a preferably conical connection 323 and/or a closure element 324, the piston 322 preferably being arranged so as to be axially movable in the housing 321, and/or it being possible for a fluid to be received, sucked in and/or dispensed via the connection 323 by means of the transfer instrument 320 by actuating the transfer instrument 320 or the piston 322.

As already explained, the treatment agent TA is preferably introduced into the transfer instrument 320 in the delivery state of the kit 300, such that the treatment agent TA can be transferred directly into the extraction instrument 340 by means of the transfer instrument 320.

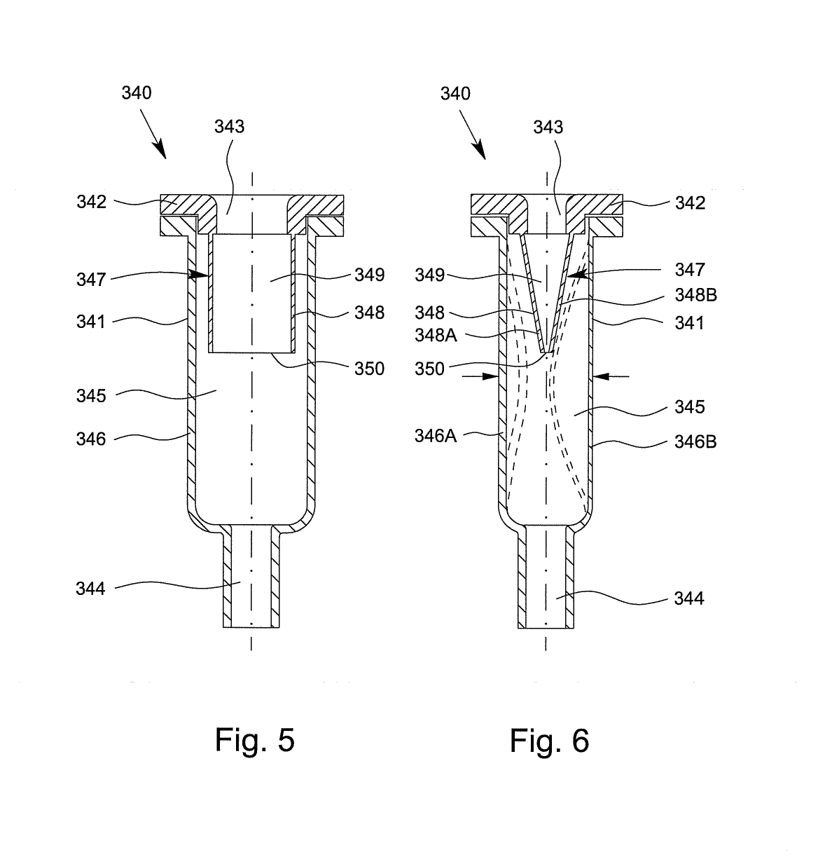

FIGS. 5 & 6 are each a schematic section through the proposed extraction instrument 340, the extraction instrument 340 being rotated in FIG. 6 by 90° about the longitudinal axis of the extraction instrument 340 in comparison with FIG. 5.

The extraction instrument 340 is preferably designed to pretreat the sample P. In particular, the sample P can be pretreated or prepared for the test of the sample P by means of the extraction instrument 340 and/or in the extraction instrument 340.

The extraction instrument 340 is preferably designed to receive the receiving instrument 310 or the receiving element 311 at least in part. In particular, the receiving element 311 can be introduced at least in part into the extraction instrument 340, in particular in order to pretreat the sample P received by means of the receiving element 311, particularly preferably in order for the analytes of the sample P to be dissolved out of or extracted from said sample and/or activated in particular by cell disruption.

In particular, the extraction instrument 340 makes it is possible for the receiving instrument 310, in particular the receiving element 311 of the receiving instrument 310, to be acted upon mechanically, in particular in order to compress the receiving element 311 in the extraction instrument 340, and/or such that the receiving element 311 is wrung out or squeezed in the extraction instrument 340, and/or such that the sample P is released at least in part from the receiving element 311.

The extraction instrument 340 is preferably elongate and/or the cross section thereof is preferably at least substantially elliptical or circular.

The extraction instrument 340 preferably comprises a main body 341, the main body 341 preferably being formed in one piece. In particular, the main body 341 is made of and/or injection-molded from plastics material.

The extraction instrument 340 preferably comprises an insert 342, the insert 342 preferably being inserted, plugged or clipped into the main body 341 at least in part. The insert 342 is preferably made of and/or injection-molded from plastics material.

Particularly preferably, the insert 342 is connected or connectable to the main body 341 in a form-fitting, interlocking, force-fitting and/or bonded manner, in particular with a press fit, tight fit or interference fit.

The extraction instrument 340 is therefore preferably formed in multiple parts, in particular in two parts. However, other structural solutions are also possible in which the extraction instrument 340 is formed in one piece or forms a unit.

The extraction instrument 340 preferably comprises an inlet 343, an outlet 344 and/or an extraction chamber 345, the inlet 343 and/or the outlet 344 preferably being fluidically connected to the extraction chamber 345. In particular, fluid can flow axially through the extraction instrument 340, in particular via the inlet 343, the extraction chamber 345 and the outlet 344.

The inlet 343 and the outlet 344 are preferably arranged at the axial ends of the extraction instrument 340.

Preferably, the inlet 343 is formed by the insert 342, and/or the outlet 344 is formed by the main body 341.

Preferably, the receiving instrument 310 and/or the receiving element 311 can be introduced into the extraction chamber 345 via the insert 342 and/or the inlet 343, in particular such that the receiving element 311 is arranged in the extraction chamber 345.

The outlet 344 is preferably designed as a connecting piece. In particular, the transfer instrument 320 or the connection 323 of the transfer instrument 320 can be fluidically connected to the extraction instrument 340 or the extraction chamber 345 via the outlet 344.

More particularly preferably, the transfer instrument 320 or the connection 323 can be plugged onto the outlet 344 of the extraction instrument 340.

The extraction instrument 340 or the main body 341 preferably comprises a wall 346, the wall 346 preferably defining or delimiting the extraction chamber 345 in particular laterally and/or radially.

The extraction instrument 340 or the extraction chamber 345 preferably has a volume or capacity of less than 10 ml or 5 ml, particularly preferably less than 3 ml, and/or more than 0.5 ml or 1 ml.

Preferably, the extraction instrument 340 is flexible and/or compressible at least in part, in particular in order to reduce the volume or capacity of the extraction chamber 345, and/or to have a mechanical effect on the receiving element 311 in the extraction chamber 345, in particular such that the sample P is released from the receiving element 311.

In particular, in order to make simple separation or squeezing of the receiving element 311 in the extraction chamber 345 possible, the extraction instrument 340, in particular the wall 346, preferably has a varying wall thickness, as shown in FIG. 6.

Particularly preferably, the extraction instrument 340, in particular the wall 346, comprises a first side part 346A and a second side part 346B, the first side part 346A preferably being thicker and/or more rigid than the second side part 346B.

The thickness of the first side part 346A is preferably more than 0.1 mm or 0.2 mm, particularly preferably more than 0.3 mm or 0.8 mm, and/or less than 5 mm or 3 mm.

The thickness of the second side part 346B is preferably less than 1 mm or 0.5 mm, particularly preferably less than 0.3 mm or 0.2 mm, in particular less than 0.15 mm.

The thickness of the second side part 346B is preferably less than 80% or 70%, particularly preferably less than 50% or 30%, of the thickness of the first side part 346A.

Preferably, when the extraction instrument 340 or the wall 346 is compressed or actuated, with the same force on both sides, the second side part 346B can be deflected to a greater extent than the first side part 346A, as indicated by dashed lines in FIG. 6.

As already explained at the outset, the extraction instrument 340 is preferably designed to automatically close the extraction chamber 345 and/or the inlet 343 when the extraction instrument 340 is compressed or actuated, in particular such that the extraction chamber 345 is sealed towards the inlet 343, and/or such that no fluid can leave the extraction instrument 340 or the extraction chamber 345 when the extraction instrument 340 is actuated.

The extraction instrument 340 preferably comprises at least one valve 347, the valve 347 preferably being assigned to the inlet 343, and/or being designed to seal the extraction chamber 345 from the outside or towards the inlet 343 and/or to prevent a fluid, in particular the sample P and/or the treatment agent TA, from leaving the extraction chamber 345 or via the inlet 343, when the extraction instrument 340 is actuated.

Preferably, the valve 347 can be actuated, particularly preferably closed, by compressing the extraction instrument 340 or the wall 346, in particular such that, when the extraction instrument 340 is compressed and/or during extraction, the sample P and/or the treatment agent TA cannot leave the extraction chamber 345.

The valve 347 is preferably formed by the insert 342. However, other solutions are also possible here, in particular those in which the valve 347 is formed by the main body 341 and/or the wall 346.

Preferably, the valve 347 directly adjoins the inlet 343 and/or the valve 347 extends from the inlet 343 into the extraction chamber 345.

The valve 347 is preferably elongate and/or planar and/or designed as a film valve, and/or can be compressed, or actuated or closed by being compressed.

Preferably, the valve 347 comprises, in particular, an elongate valve body 348 and/or an in particular elongate valve chamber 349, the valve body 348 preferably defining or delimiting the valve chamber 349 in particular laterally and/or radially.

Preferably, the valve body 348 and/or the valve chamber 349 is arranged within the extraction chamber 345 at least in part, particularly preferably completely.

The valve 347 preferably comprises a valve opening 350, the valve body 348 and/or the valve chamber 349 preferably tapering from the inlet 343 towards the outlet 344 and/or in the extraction chamber 345 towards the valve opening 350.

Particularly preferably, the cross section of the valve body 348 and/or the valve opening 350 is at least substantially elliptical and/or slot-like.

Preferably, the inlet 343 is fluidically connected to the extraction chamber 345 via the valve 347, in particular the valve chamber 349 and/or the valve opening 350.

The valve 347, in particular the valve body 348, is preferably flexible and/or compressible at least in part, in particular by actuating or compressing the extraction instrument 340 or the wall 346.

The valve 347 or the valve body 348 preferably comprises a first valve side 348A and a second valve side 348B, the first valve side 348A and the second valve side 348B preferably each forming or comprising a planar or flat side of the preferably planar or flat valve 347.

Preferably, the first valve side 348A is assigned to or faces the first side part 346A, and/or the second valve side 348B is assigned to or faces the second side part 346B.

Preferably, the wall 346, in particular the first side part 346A and/or the second side part 346B, can be pressed against the valve 347, in particular the valve body 348 or the valve opening 350, such that the valve 347 or the valve opening 350 closes.

As shown in particular in FIG. 6, the valve sides 346A, 346B are preferably oriented obliquely to the side parts 346A, 346B of the wall 346.

Preferably, the valve opening 350 is arranged at least substantially centrally and/or coaxially in the extraction chamber 345, and/or the longitudinal axis of the extraction instrument 340 extends through the valve 347, in particular the valve chamber 349 and/or the valve opening 350. More particularly preferably, the valve opening 350 is arranged to be at least substantially equidistant from the first side part 346A and the second side part 346B, at least when the extraction instrument 340 is not actuated.

In an alternative embodiment (not shown), the valve opening 350 is preferably arranged closer to the second side part 346B than to the first side part 346A. In an embodiment of this kind, it may be provided that the second valve side 348B extends at least substantially in parallel with the second side part 346B. Therefore, in an embodiment of this kind, the wall 346 or the second side part 346B only has to be deflected to a slight extent in order to close the valve 347 or the valve opening 350. This allows a particularly rapid closure of the valve 347 when the extraction instrument 340 is compressed or actuated.

Preferably, the receiving instrument 310 can be inserted into the extraction instrument 340 or the extraction chamber 345 via the inlet 343 and through the valve 347, in particular the valve chamber 349 and/or the valve opening 350.

In particular, the valve 347 or the valve body 348 is designed to be sealingly positioned against the receiving instrument 310 or the connection element 312 when the extraction instrument 340 or the wall 346 is compressed, in particular such that the extraction chamber 345 is sealed towards the inlet 343 or from the outside.

Optionally, the extraction instrument 340 comprises a filter (not shown), the filter preferably being integrated in the extraction instrument 340 and/or arranged in or on the outlet 344. This makes it possible for the analytes of the sample P to be extracted or dissolved out as well as to be filtered by means of the extraction instrument 340, in particular such that the test can be carried out immediately after the sample P that has been pretreated or prepared in this way has been removed, in particular without any further pretreatment of the sample P.

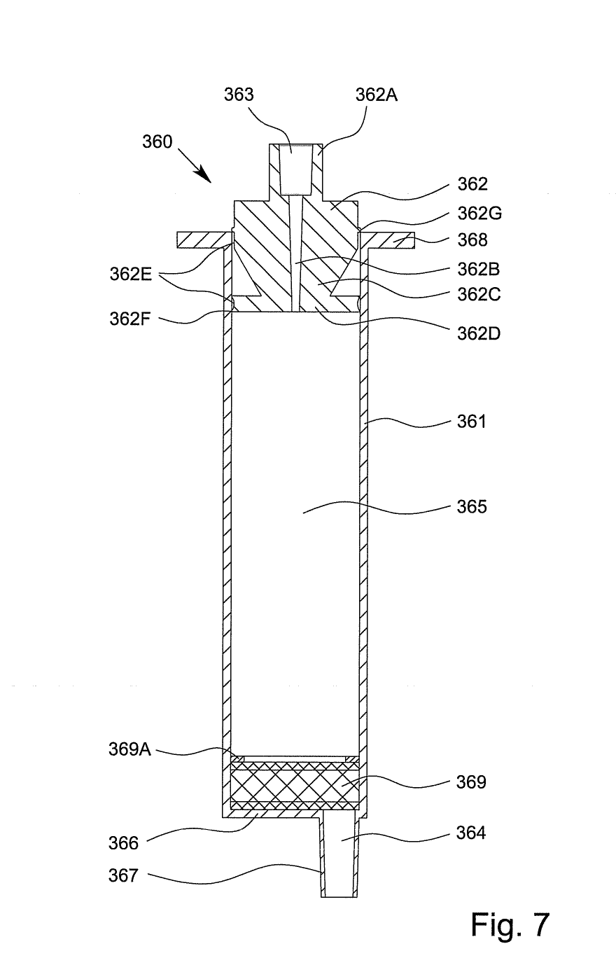

FIG. 7 is a schematic section through the proposed dispensing instrument 360.

The dispensing instrument 360 is preferably designed for (further) pretreatment or preparation of the sample P that has in particular already been pretreated or prepared via the extraction instrument 340.

Preferably, the dispensing instrument 360 is designed to (directly) dispense the pretreated sample P to the cartridge 100, the sample P preferably being (further) prepared or pretreated, in particular filtered, by means of or during dispensing of the sample P, as described in greater detail in the following.

Preferably, the dispensing instrument 360 is, in principle, constructed as a syringe.

Preferably, the dispensing instrument 360 comprises an in particular cylindrical housing 361 and a piston 362, the piston 362 preferably being axially movable in the housing 361, in particular in order to dispense the sample P.

The dispensing instrument 360 preferably comprises an inlet 363, an outlet 364 and/or a chamber 365, the inlet 363 preferably being fluidically connected to the outlet 364 by means of the chamber 365, and/or fluid being able to flow through the chamber 365 from the inlet 363 to the outlet 364.

Preferably, the inlet 363 is formed by the piston 362 and/or the piston 362 comprises the inlet 363, as described in greater detail in the following.

The dispensing instrument 360 is preferably elongate and/or fluid can preferably flow axially therethrough, in particular from the inlet 363 to the outlet 364.

Preferably, the dispensing instrument 360 comprises a base 366, the outlet 364 preferably being arranged on or in the base 366 of the dispensing instrument 360.

In the embodiment shown, the outlet 364 is preferably formed by an in particular conical connecting piece 367, in particular on the base 366, the outlet 364 and/or connecting piece 367 preferably being arranged eccentrically or in the edge region. However, other solutions are also possible here.

Preferably, the dispensing instrument 360, in particular the housing 361, comprises a holder 368 for holding the dispensing instrument 360, in particular when the dispensing instrument 360 is actuated, the holder 368 preferably being formed by a collar or two opposing radial projections at the axial end of the housing 361.

Preferably, the dispensing instrument 360 comprises an in particular integrated filter 369, the filter 369 preferably being designed to filter the sample P, in particular to separate out undesired sample components, such as particles, impurities, proteins or the like, and/or to let through nucleic-acid sequences as analytes of the sample P.

Particularly preferably, the filter 369 is arranged on the base 366 and/or on or directly upstream of the outlet 364, in particular such that the sample P flows through the filter 369 or is filtered by means of the filter 369 immediately before dispensing.

Preferably, the filter 369 is connected to the housing 361 and/or fastened to the base 366 in a form-fitting, interlocking, force-fitting and/or bonded manner. In the embodiment shown, the dispensing instrument 360 preferably comprises a securing element 369A, the securing element 369A preferably being designed to axially secure the filter 369. The securing element 369A can, for example, be designed as a securing ring, in particular a snap ring, in order to axially secure the filter 369 or axially hold said filter in position. However, other solutions are also possible here.

The chamber 365 is preferably defined or delimited by the housing 361 in particular laterally and/or radially and is defined or delimited by the piston 362 and the filter 369 in particular axially.

Preferably, the volume of the chamber 365 can be reduced by actuating the dispensing instrument 360 and/or by axially moving the piston 362 towards the outlet 364 or the filter 369.

Preferably, the dispensing instrument 360, in particular the chamber 365, has, in the delivery state or in the unactuated state, as shown in FIG. 7, a maximum volume of more than 2 ml or 5 ml, particularly preferably more than 10 ml, and/or less than 100 ml or 50 ml.

Preferably, the piston 362 can be moved axially as far as the filter 369 or the base 366 and/or can be moved in the housing 361 towards the outlet 364 until the piston 362 pushes against the filter 369, the base 366 and/or the optional securing element 369A.

The piston 362 is preferably formed in one piece and/or forms a unit. The piston 362 is particularly preferably made of and/or injection-moulded from plastics material.

Preferably, a fluid can flow axially through the piston 362, in particular such that a fluid and/or the sample P can be introduced into the chamber 365 via the piston 362.

As already mentioned, the inlet 363 to the chamber 365 is preferably formed by the piston 362 or integrated in the piston 362.

Particularly preferably, the piston 362 comprises a connection 362A, the connection 362A preferably being designed as a connecting piece and/or being arranged on the side of the piston 362 that is remote from the chamber 365. In particular, the transfer instrument 320 can be fluidically linked to the piston 362 or the connection 362A, or can be connected to the piston 362 or the connection 362A.

Preferably, the piston 362 comprises a piston channel 362B, the piston channel 362B preferably extending axially through the piston 362, in particular from the connection 362A to the chamber 365, and/or such that a fluid can be introduced into the chamber 365 through the piston 362.

In the embodiment shown, the piston channel 362B tapers, preferably towards the chamber 365. However, other solutions are also possible here, in particular those in which the piston channel 362B has an at least substantially constant flow cross section.

The piston 362 is preferably formed by a main body comprising a plurality of ribs 362C, the ribs 362C preferably extending radially outwards or towards the housing 361.

Preferably, the piston 362 comprises an in particular plate-shaped piston head 362D, the piston head 362D preferably forming an axial end of the piston 362 and/or being arranged on the side of the piston 362 that faces the chamber 365.

The piston 362 is preferably radially guided in the housing 361. In particular, the piston 362 comprises a guide 362E, the guide 362E preferably being formed by the ribs 362C and/or the piston head 362D.

Preferably, the piston 362 comprises a seal 362F, the seal 362F preferably being formed by the piston head 362D and/or being designed to seal the gap between the piston 362, in particular the piston head 362D, and the housing 361, in particular such that no fluid can flow between the piston 362, in particular the piston head 362D, and the housing 361.

Optionally, the piston 362 is provided with a securing element 362G, the securing element 362G preferably being designed to prevent the piston 362 from being unintentionally pushed into the housing 361, for example when the dispensing instrument 360 is being transported and/or when the transfer instrument 320 is being connected. Particularly preferably, the securing element 362G is designed as a preferably peripheral projection on the piston 362, as shown in FIG. 7.

By means of the piston 362, it is therefore possible to fluidically connect the transfer instrument 320 to the chamber 365, to introduce the sample P into the chamber 365 through the piston 362, to increase the pressure in the chamber 365 by (subsequently) actuating the dispensing instrument 360 or moving the piston 362 towards the outlet 364 or the filter 369, and/or to filter the sample P introduced into the chamber 365 by moving the piston 362 towards the outlet 364 or the filter 369, to push said sample through the filter 369, and/or to dispense said sample in particular to the cartridge 100 via the outlet 364.

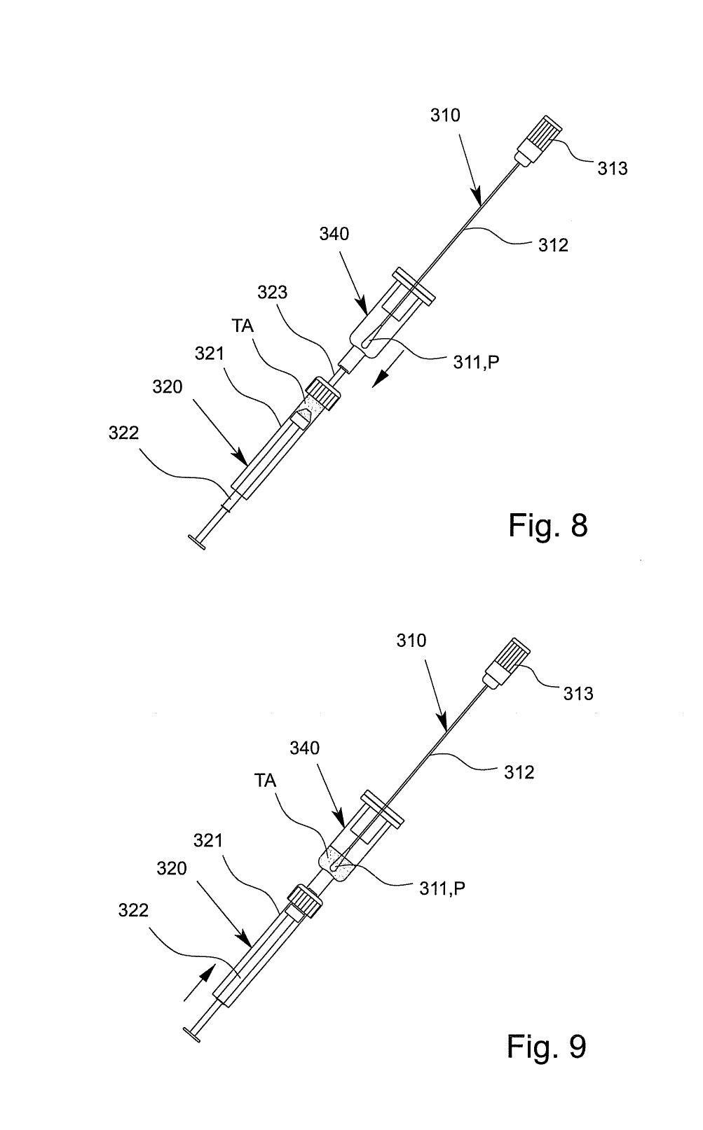

In the following, the proposed method is described in greater detail with reference to FIG. 8 to FIG. 13.

By means of the proposed method, the sample P is preferably pretreated or prepared for a subsequent, in particular molecular-biological test, preferably by means of the cartridge 100 and/or the analysis device 200, in particular such that the sample P that has been pretreated in this way can be tested or introduced into the cartridge 100, directly and/or without any further treatment steps.

In particular, by means of the proposed method, analytes, particularly preferably nucleic-acid sequences, are dissolved, extracted, eluted, activated and/or filtered from the sample P, in particular in order to subsequently detect or identify the analytes or nucleic-acid sequences, in particular electrochemically, preferably by means of the cartridge 100 and/or the analysis device 200.

Preferably, the proposed method and/or individual method steps of the proposed method, some of the method steps of the proposed method or all of the method steps of the proposed method is/are carried out by means of the proposed kit 300, the receiving instrument 310, the transfer instrument 320, the extraction instrument 340 and/or the dispensing instrument 360.

Preferably, in a first method step, the sample P is removed, in particular from a human or animal, particularly preferably by means of the receiving instrument 310 or the receiving element 311.

Particularly preferably, a swab, in particular a mucosa swab, more particularly preferably a nasal mucosa swab, is taken for this purpose by means of the receiving instrument 310, sample material preferably being received or absorbed by the receiving element 311.

Preferably, the sample P that has in particular been obtained in this way is pretreated or prepared, in particular by means of the extraction instrument 340 and/or the dispensing instrument 360, before the actual test is carried out.

Preferably, the sample P, the receiving instrument 310, the receiving element 311 and/or the treatment agent TA is introduced into the extraction instrument 340 and/or the extraction chamber 345 in a further, preferably second, method step.

Particularly preferably, the receiving instrument 310 and/or the receiving element 311 is inserted at least in part into the extraction chamber 345 via the inlet 343 and/or through the valve 347.

Preferably, the extraction instrument 340, when in use, is oriented or held so as to be substantially vertical and/or such that the inlet 343 is arranged at the top and the outlet 344 is arranged at the bottom. In particular, the receiving instrument 310 or the receiving element 311 is introduced into the extraction chamber 345 from above in the normal operating position and/or the treatment agent TA is introduced into the extraction chamber 345 from below in the normal operating position.

As shown in FIG. 8, the treatment agent TA is preferably introduced into the extraction instrument 340 or the extraction chamber 345 by means of the transfer instrument 320, in particular via the outlet 344, particularly preferably after the receiving instrument 310 or the receiving element 311 has been introduced into the extraction instrument 340 or the extraction chamber 345. However, other solutions are also possible here, in particular those in which the treatment agent TA is first introduced into the extraction instrument 340 or the extraction chamber 345 and only then is the receiving instrument 310 or the receiving element 311 inserted into the extraction instrument 340 or the extraction chamber 345. In particular, method variants are also possible in which the treatment agent TA is already introduced into the extraction instrument 340 or the extraction chamber 345 at the factory.

Preferably, in a further, preferably third, method step, the sample P is lysed and/or the analytes, in particular nucleic-acid sequences, are dissolved out of or extracted from the sample P in the extraction instrument 340, eluted and/or activated by cell disruption.

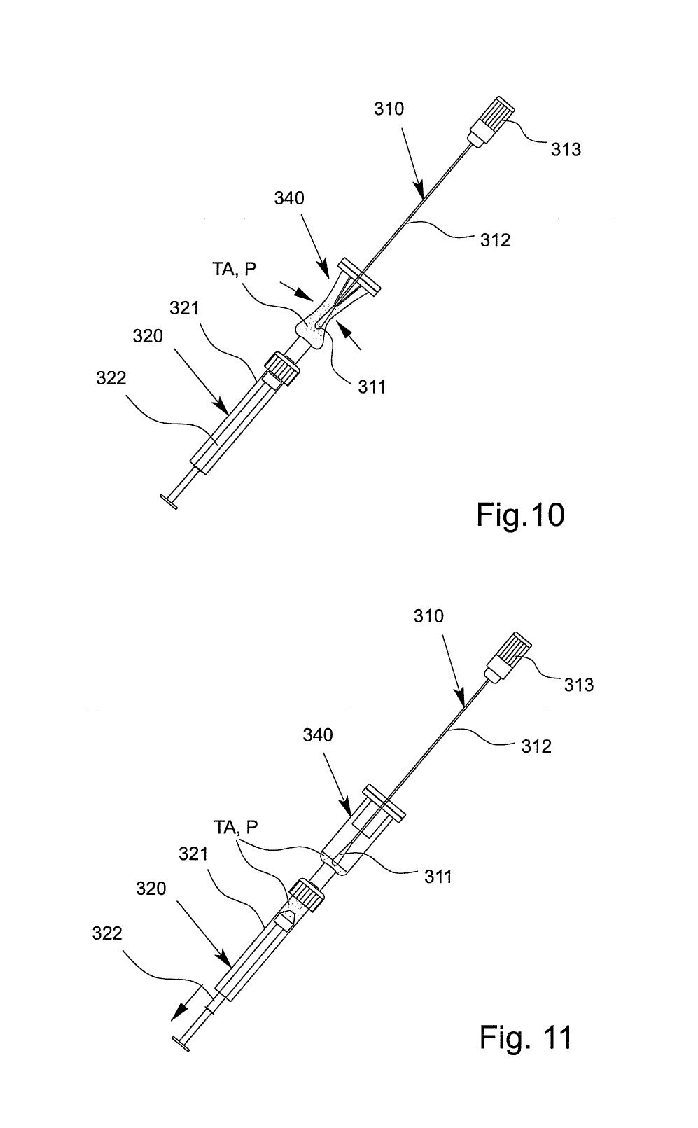

As shown in particular in FIG. 10, the extraction instrument 340, in particular the wall 346, is compressed in order to extract the sample P, in particular such that the wall 346, particularly preferably the first side part 346A and/or the second side part 346B, exerts pressure on the receiving instrument 310 or the receiving element 311, and/or such that the receiving instrument 310 or the receiving element 311 is squeezed or wrung out, and/or such that the sample P is released from the receiving element 311 at least in part. In other words, by compressing or actuating the extraction instrument 340, the receiving element 311 in the extraction instrument 340 is therefore mechanically acted upon, such that the sample P is separated from the receiving element 311. The structural design of the extraction instrument 340 as described above allows a particularly simple and hygienic extraction or release of the sample P and/or the sample components.

In particular, in order to prevent the sample P and/or the treatment agent TA from leaving or spurting out of the extraction instrument 340 during extraction, it is preferably provided that the extraction instrument 340 or the valve 347 of the extraction instrument 340 is closed by actuating the extraction instrument 340 or the wall 346.

Particularly preferably, by compressing the extraction instrument 340, the wall 346 of the extraction chamber 345 is pressed against the valve 347 and/or the valve body 348 such that the valve opening 350 is closed and/or the valve body 348, in particular the first valve side 348A and/or the second valve side 348B, sealingly abuts the receiving instrument 310 or the connection element 312 of the receiving instrument 310. This results in the extraction chamber 345 being closed or sealed from the outside or towards the inlet 343.

Preferably, in a subsequent, preferably fourth, method step, the sample P that has been pretreated in this way is removed from the extraction instrument 340, in particular by means of the transfer instrument 320, via the outlet 344 of the extraction instrument 340, as shown in FIG. 11. Other method variants are however also possible, in particular those in which the pretreated sample P is supplied directly from the extraction instrument 340 to the dispensing instrument 360 and/or the cartridge 100.

Preferably, in a further, preferably fifth, method step, the sample P is dispensed to or into the cartridge 100, in particular by means of the dispensing instrument 360.

For this purpose, the dispensing instrument 360, in particular the connecting piece 367, is preferably fluidically connected to the cartridge 100, in particular the receiving cavity 104 of the cartridge 100. Particularly preferably, the connecting piece 367 of the dispensing instrument 360 is inserted into the connection 104A of the cartridge 100.

In order to receive the sample P, the cartridge 100 is preferably oriented horizontally and/or laid flat, for example on a table, in particular such that the connection 104A of the cartridge 100 points upwards and/or is accessible from above.

Particularly preferably, the dispensing instrument 360 is oriented vertically and/or connected to the cartridge 100 or inserted into the connection 104A from above.

Preferably, the transfer instrument 320 is oriented vertically and/or fluidically connected to the dispensing instrument 360, in particular plugged into the connection 362A.

Preferably, the sample P is inserted into the dispensing instrument 360, in particular by means of the transfer instrument 320, particularly preferably through the piston channel 362B of the piston 362 and/or from above, in particular such that the sample P collects at the bottom or on the base 366 of the dispensing instrument 360 and/or on the filter 369.

Particularly preferably, the sample P is first completely introduced into the dispensing instrument 360 or the chamber 365, before the piston 362 is moved axially. Other method variants are however also possible here, in particular those in which, while the sample P is still being introduced into the dispensing instrument 360, the piston 362 is already moved axially and/or the sample P is filtered and/or dispensed to the cartridge 100.

By moving the piston 362 towards the filter 369, the outlet 364 and/or downwards, the pressure in the chamber 365 is preferably increased, in particular until the pressure resistance of the filter 369 is overcome, the sample P is pushed through the filter 369 and/or the sample P is filtered by means of the filter 369.

The sample P is therefore preferably filtered, in particular by means of the integrated filter 369, while or immediately before the sample P is dispensed to the cartridge 100, the pressure required for filtration preferably being created in the chamber 365 by actuating the dispensing instrument 360 or the piston 362.

Preferably, the piston 362 is completely pressed down and/or against the filter 369, in particular such that almost all of the sample P is dispensed from the dispensing instrument 360, as shown in FIG. 13.

Preferably, the transfer instrument 320 and/or the dispensing instrument 360 is then (together) separated from the cartridge 100 and/or disposed of.

After the sample P has been received, the cartridge 100 is preferably closed in a liquid-tight and/or gas-tight manner by means of the closure element 130, in particular such that the cartridge 100 together with the sample P can be inserted into the analysis device 200 for the subsequent test.

Individual aspects and features of the present invention and individual method steps and/or method variants may be implemented independently from one another, but also in any desired combination and/or order.

What is claimed is:

1. A method for pretreating a biological sample for testing of the sample, comprising:
receiving the sample in a receiving element,
inserting the receiving element at least in part into an extraction instrument that is compressible at least in part,
separating the sample being from the receiving element by means of a treatment agent, the method further comprising at least one of the following steps:
closing the extraction instrument being by compressing the extraction instrument to separate the sample from the receiving element,
introducing the treatment agent into the extraction instrument by means of a transfer instrument,
removing the sample from the extraction instrument by means of the transfer instrument, and
introducing the sample into a dispensing instrument and filtering the sample in the dispensing instrument.

2. The method according to claim 1, wherein the extraction instrument comprises an extraction chamber and a wall for defining or delimiting the extraction chamber, the wall being elastically deformable at least in part, and wherein the extraction instrument has at least one of the following features:
the extraction instrument comprising at least one valve that is actuated by the valve compressing the wall, and
the wall comprising a first side part and a second side part, the first side part being more rigid than the second side part.

3. The method according to claim 1, wherein the dispensing instrument comprises a housing and a piston which is moveable axially in the housing to dispense the sample, and wherein the dispensing instrument has at least one of the following features:
the piston has a piston channel which extends axially through the piston, and
the dispensing instrument comprises a filter for filtering the sample.

4. Method according to claim 1, wherein the sample is removed from the extraction instrument by means of the transfer instrument and is delivered to a dispensing instrument or cartridge for filtering or testing.

5. Method according to claim 1, wherein a valve of the extraction instrument is closed by compressing the extraction instrument.

6. Method according to claim 1, wherein the dispensing instrument is fluidically coupled to a cartridge for testing the sample before the sample is received or filtered.

7. Method according to claim 1, wherein the sample is introduced into the dispensing instrument through a piston of the dispensing instrument.

8. Method according to claim 1, wherein the sample is filtered or dispensed through a filter by actuating the dispensing instrument.

9. Method according to claim 1, wherein saliva or mucosa from animals is pretreated as the sample for subsequent testing.

10. Method according to claim 1, wherein nucleic-acid sequences are extracted or filtered from the sample.

11. Method according to claim 1, wherein the treatment agent is introduced into the extraction instrument via an outlet of the extraction instrument by means of the transfer instrument and wherein the sample is removed from the extraction instrument via the outlet by means of the transfer instrument.

12. Extraction instrument for extracting a biological sample from a receiving element, the extraction instrument comprising:
an extraction chamber and a wall for defining or delimiting the extraction chamber, the wall being elastically deformable at least in part, and
the extraction instrument having at least one of the following features:
the extraction instrument has at least one valve which is actuatable for compressing the wall, and
the wall comprising a first side part and a second side part, the first side part being more rigid than the second side part.

13. Extraction instrument according to claim 12, wherein the valve is a film valve.

14. Extraction instrument according to claim 12, wherein the valve comprises an at least substantially planar valve body having at least one of the following features: the cross section of the valve body being at least substantially elliptical, it being possible for the valve body to be compressed by compressing the extraction instrument or the wall, and the valve body extending into the extraction chamber.

15. Extraction instrument according to claim 12, wherein the extraction chamber has an elliptical cross section.

16. Extraction instrument according to claim 12, wherein the first side part and the second side part each comprise or form a flat side of the wall, and/or wherein the first side part and the second side part can be pressed against the valve such that the valve closes.

17. Dispensing instrument for dispensing a biological sample to a cartridge, the dispensing instrument comprising:
a housing and
a piston, the piston being movable axially in the housing to dispense the sample, and
the dispensing instrument having at least one of the following features:
the piston comprising a piston channel which extends axially through the piston, and
the dispensing instrument comprising a filter for filtering the sample.

18. Dispensing instrument according to claim 17, wherein the sample is introducable into the dispensing instrument through the piston channel.

19. Dispensing instrument according to claim 17, wherein the piston comprises or forms a connection for a transfer instrument.

20. Dispensing instrument according to claim 17, wherein the dispensing instrument comprises an outlet, the filter being arranged on or directly upstream of the outlet, and/or wherein the filter is arranged in the housing.

21. Dispensing instrument according to claim 17, wherein the dispensing instrument comprises a chamber, the piston and the filter defining or delimiting the chamber in an axial direction.

22. Kit for pretreating a biological sample for testing of the sample, the kit comprising:
a receiving instrument having a receiving element for receiving the sample,
an extraction instrument for extracting the sample from the receiving element, and
a dispensing instrument for dispensing the sample to a cartridge,
wherein the extraction instrument comprises an extraction chamber and a wall for defining or delimiting the extraction chamber, the wall being elastically deformable at least in part, and/or
wherein the dispensing instrument comprises a housing and a piston which is moveable axially in the housing in order to dispense the sample.

23. Kit according to claim 22, wherein the extraction instrument comprises at least one valve for compressing the wall.

24. Kit according to claim 22, wherein the wall comprises a first side part and a second side part, the first side part being more rigid than the second side part.

25. Kit according to claim 22, wherein the piston comprises a piston channel which extends axially through the piston.

26. Kit according to claim 22, wherein the dispensing instrument comprises a filter for filtering the sample.

27. Kit according to claim 22, wherein the kit further comprises at least one of a transfer instrument for introducing the sample into the dispensing instrument, and a treatment agent for treating the sample.

28. Kit according to claim 27, wherein the treatment agent is or comprises a lysis buffer for cell disruption.
